(19) United States
(12) Patent Application Publication
(10) Pub. No.: US 2015/0173598 A1
(43) Pub. Date: Jun. 25, 2015

(54) Intubating Airway

(71) Applicant: Mark Alexander, Bloomington, MN (US)

(72) Inventor: Mark Alexander, Bloomington, MN (US)

(21) Appl. No.: 14/135,285

(22) Filed: Dec. 19, 2013

(51) Int. Cl. A61B 1/267; A61B 1/00; A61M 16/04; A61B 1/015; A61B 1/05

(52) U.S. Cl. CPC A61B 1/267; A61B 1/015; A61B 1/053; A61M 16/0488; A61B 1/00165

(57) ABSTRACT

The intubating airway of the present invention has a shape of a long curved spatula with two channels underneath. The present invention its design, size, shape and adjustable depth of insertion provide it with unique ability to open the airway tract completely, reliably and consistently from the mouth to the larynx. This ability makes it a multifunction device: It can relieve any degree of airway obstruction when all available airway devices have failed. It convert fiber optic intubation and optical stylet intubation from difficult, time consuming and need a lot of experience into quick, easy and simple even by first time user could intubate with high success rate. It facilitates lighted stylet intubation and the intubation of a double lumen tube or nasal tube when it is difficult to intubate, and also facilitate the insertion of a TEE probe or gastroscope or bronchoscope.

### REFERENCE TO RELATED APPLICATIONS

This patent application claims the benefit of U.S. Provisional Application No. 61/958,761 filed on Aug. 5, 2013, the disclosure of which is incorporated herein by reference in its entirety.

### BACKGROUND OF THE INVENTION

1. Field of the Invention

The present invention relates to a medical device and more particularly to an intubating airway not only functions as an oropharyngeal airway which is specially designed to relieve severe airway obstruction but with added features to facilitate the process of fiber optic tracheal intubation, optical stylet intubation and to deliver local anesthetics under direct vision to numb (anesthetize) the airway tract prior to awake intubation.

The intubating airway has a curved lingual (tongue) surface which is in contact with the tongue surface of a human being, and a pharyngeal surface which is in contact with the posterior pharyngeal wall (back of throat). It has a wall in the middle and is perpendicular to both surfaces whereby forming an open sided endotracheal tube conduit to the right and a closed sided conduit to the left. Said lingual surface has a handle extended from its proximal end at an angle and a tapered distal end with a rounded atraumatic tip. The wall to the right of said endotracheal tube conduit is partially open along its full length. This allows the intubating airway to be peeled away from the endotracheal tube and removed from the patient's mouth after trachea intubation without disturbing the endotracheal tube.

It has an injection channel also referred to as suction channel embedded in the wall between the right and left conduits which give it the following added functions:
a. It can be used to deliver local anesthetics quickly and precisely under direct vision to numb (anesthetize) the airway tract from the mouth to the carina prior to awake intubation;
b. deliver oxygen to the glottis opening and prevent fogging of the scope lens; and
c. suck secretion or blood from the airway tract to prevent its aspiration into the lung and prevent it from obscuring the viewing lens of the fiber optic scope or the video camera improving visualization of the airway tract

Tracheal intubation is the placement of a flexible plastic tube into the trachea (windpipe) to maintain an open airway. It is frequently performed in critically injured, ill, or anesthetized patients to facilitate ventilation of the lungs, including mechanical ventilation, and to prevent the possibility of aspiration or airway obstruction. The most widely used route is from mouth to larynx, in which an endotracheal tube is passed through the mouth and glottis into the trachea.

Fiber optic intubation needs an open airway tract from the mouth to the larynx to be able to see the larynx and intubate.

However, the flexible thin and soft fiber optic scope is hard to maneuver, because It can't separate tissues and open the airway tract therefore an intubating airway is needed to ease the difficulty associated with fiber optic intubation.

2. Description of Related Art

The operator of the fiber optic scope needs an open airway tract from the mouth to the larynx to be able to visualize the larynx, which makes it easy for him to advance the fiber optic scope under a clear direct vision into the larynx for a successful tracheal intubation. However, the flexible thin and soft fiber optic scope is hard to maneuver because it can't separate tissues and open the airway tract. Therefore, an intubating airway is needed to ease the difficulty associated with fiber optic intubation.

Structurally, all related prior art, specifically intubating airways are composed of a spatula (tongue retractor) and one conduit underneath, or a curved tube which act as a guide or passage for the fiber optic scope. None of them can open completely the airway tract from the mouth to the larynx consistently and reliably especially in anesthetized paralyzed patients (unexpected difficult intubation) leaving the distal part of the airway tract collapsed and obstructed and the soft thin fiber optic scope can't open. Our present invention has a—C shaped spatula (tongue retractor) and 2 conduits underneath, plus an injection/suction channel. It is of a different size and shape allowing it to easily open the airway tract completely from the mouth to the larynx consistently and reliably in either spontaneously breathing or paralyzed patients. This gives it the following functional advantages over prior arts:
1—It can relieve any degree of airway obstruction including the severe one when all the available airway devices and maneuvers (oral, nasal airways, LMA, jaw thrust, head lilt, etc) have failed. It can be life saving.
2—With prior arts, fiber optic intubation is difficult, time consuming, and has a low success rate. It needs a lot of experience and can't be used in real emergencies. Our present invention makes fiber optic intubation quick, easy and simple, so that even the first time user could intubate with a high success rate. It can also be used in all situations including a real emergency. Again it can be life saving.
3—It facilitates optical stylet intubation and difficult double lumen tube intubation or difficult nasal intubation when conventional devices have failed.
4—It facilitates the insertion under direct vision of a TEE probe or endoscopes of the respiratory or gastrointestinal tracts.

U.S. Pat. No. 4,338,930 discloses an intubating airway adapted to function as a guide and passage way for an endotracheal tube for intubation of a person's trachea. However, the above intubating airway fails to open the airway fully leaving the distal part of airway tract closed and obstructed.

Other brands, such as Ovassapian, Williams, Berman, also fail to open the airway tract all the way to the larynx, leaving the distal part of the airway collapsed and obstructed and a soft thin fiber optic scope can't open the airway.

Therefore, an airway which can open the patient's airway tract from the mouth to larynx completely is desirable. The operator could see the larynx instantly and advance the fiber optic under clear and direct vision. Even the first time user could intubate with a high successful rate.

In addition the present invention has an injection/suction channel which give it the following advantages over prior arts:
5—It can be used to deliver local anesthetic quickly, precisely and efficiently under direct vision to numb (anesthetize) the airway tract from the mouth to the carina prior to awake intubation.
6—It can be used to deliver oxygen directly to the glottis opening to treat or prevent hypoxia and prevent fogging of the scope lens.
7—It can be used to suck and clear secretion, or blood from airway tract to prevent its aspiration into the patient's lung and prevent it from covering the viewing lens of the fiber optic scope or a video camera for better visualization of the laryngeal structure.

Therefore an injection/suction channel is desirable.

Examples of the commercially available intubating airways: Ovassapian U.S. Pat. No. 5,024,218; Williams U.S. Pat. No. 4,338,930; Berman U.S. Pat. No. 4,054,135; and others that disclose oral airways like: Isenberg U.S. Pat. No. 7,866,313 B2; Flam U.S. Pat. No. 5,590,643; Henry U.S. Pat. No. 5,368,016; Raimo U.S. Pat. No. 3,306,298 A; Sheffer U.S. Pat. No. 3,543,751 A; Alfery U.S. Pat. No. 6,196,244 B.

Furthermore, if blood or secretion stays in the air tract, it will cover the fiber optic lens and impair visualization of the laryngeal structure. The above mentioned intubating airways do not provide a means for removal of blood and secretion. Therefore, a suction channel is needed to perform this function.

### SUMMARY OF THE INVENTION

The intubating airway has the shape of a C-shaped spatula (tongue retractor) with 2 conduits underneath: a right open sided endotracheal conduit and a left close sided conduit which can accommodate a video camera. It has a built in suction/injection channel.

The intubating airway has a curved lingual (tongue) surface which is in contact with the tongue surface of a human being, and a pharyngeal surface which is in contact with the posterior pharyngeal wall (back of throat). Said lingual surface has a handle extended from its proximal end at an angle and a tapered distal end with a rounded atraumatic tip. The wall to the right of said endotracheal tube conduit is partially open along its full length. it allows the airway to be peeled away from the endotracheal tube and be removed from the patient's mouth after trachea intubation without disturbing the endotracheal tube. An injection channel, also referred to as suction channel, runs through said wall along its full length, having a proximal end and a distal end. The present invention design, size and shape provide it with the unique ability to open the airway tract consistently and reliably but more importantly to open the airway tract completely from the mouth to the larynx. This ability makes it a multifunction device:

a. it can relieve severe airway obstruction;

b. makes fiber optic intubation quick and easy;

c. facilitates optical stylet intubation and the intubation of double lumen tube or nasal intubation when it is difficult;

d. and also facilitate the insertion of TEE probe or endoscopes of the respiratory or gastro intestinal tract under direct vision.

It has a suction/injection channel embedded in the wall between the right and left conduits which give it the following added functions;

a. It can be used to deliver local anesthetics quickly and precisely under direct vision to numb the airway tract from the mouth to the carina prior to awake intubation;

b. to deliver oxygen directly to the glottis opening or to suck secretions or blood from the airway tract to prevent its aspiration and improve visualization of the fiber optic scope or the video camera.

In an exemplary embodiment of the present invention, there is disclosed an intubating airway, which can relieve severe life threatening airway tract obstruction. Its adjustable depth of insertion makes it adapt and fit any size of patient airway tract. It also allows controlled mask ventilation and oxygenation

It is adapted to guide the flexible, thin fiber optic tube during the process of intubation, thus converts fiber optic intubation from a time consuming low success rate that can't be used in real emergency into a rapid, successful intubation modality that can be used in all situations including a real emergency. With its injection/suction channel it can be used to deliver local anesthetics quickly and precisely under direct vision to numb (anesthetize) the airway tract from the mouth to the carina prior to awake intubation. It facilitates optical stylet intubation and difficult double lumen tube intubation or difficult nasal intubation when conventional devices have failed. It functions as a guide to facilitate the insertion of such devices as endoscopes into upper respiratory tract or gastro intestinal tracts or TEE probe under direct vision. In addition, its injection/suction channel can be used to deliver oxygen directly to the glottic opening to treat or prevent hypoxia and to suck and clear secretion or blood from the airway tract to prevent its aspiration into the lung and prevent it from covering the viewing lens of fiber optic scope or video camera.

The intubating airway has a curved lingual (tongue) surface which is in contact with the tongue surface of a human being, and a pharyngeal surface which is in contact with the posterior pharyngeal wall (back of throat).

Said lingual surface has a handle extended from its proximal end at an angle and a tapered distal end with a rounded traumatic tip.

Underneath said lingual surface, a wall stands between said lingual surface and a pharyngeal surface perpendicularly to both lingual surface and pharyngeal surface, whereby forming an endotracheal tube conduit on one side, preferably on the right side; and a close-sided channel on the other side, preferably on the left side (left side conduit). An injection channel, also referred to a suction channel, runs through said wall along its full length, having a proximal end and a distal end. Said close-sided conduit is an empty, closed channel having a closed distal end and an open proximal end, which can accommodate a video camera. Said handle extends from said proximal end towards said pharyngeal wall. Said endotracheal tube conduit runs approximately the proximal ⅔ along said lingual surface. The wall to the right of said endotracheal tube conduit is partially open along its full length. This allows operator to peel away the airway from the endotracheal tube after intubation and remove it from the patient's mouth without disturbing the tube.

The left over exterior side wall forms a rim extending upwardly from said pharyngeal surface. The distal of said lingual surface is free and makes the distal tapered end of the airway intubator with rounded atraumatic tip. In fact, the intubating airway has a shape of long curved spatula with two channels underneath said lingual surface.

In the first modified alternative embodiment of the present invention, the intubating airway has only the left closed sided conduit, which will accommodate a video camera and then it will function as a video laryngoscope. There will be no right endotracheal conduit. It will be used to facilitate optical stylet intubation and in difficult intubation of double lumen or difficult nasal intubation. Also it will facilitate the insertion under direct vision of TEE probe or endoscopes, for example gastroscopes, bronchoscopes, etc. With the built in injection/suction channel it can deliver local anesthetics quickly and precisely to numb (anesthetize) the airway tract from the mouth to the carina under direct vision prior to awake intubation.

In the second modified alternative embodiment of the present invention the airway instead of having a C shape, it will be L shaped and is right angled. It will facilitate in the difficult intubation of patients with certain unusual anatomy of their airway tract.

In the third modified alternative embodiment of the present invention the suction (injection channel) is located in the middle and embedded in the underneath of the lingual surface and extend from the proximal end to almost the distal end of the airway.

In the fourth modified alternative embodiment of the present invention the airway handle will extend to either sides or to the front.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a prospective view of an exemplary intubating airway of present invention;

FIG. 2 is a right side view of an exemplary intubating airway of present invention;

FIG. 3 is a prospective view of an exemplary intubating airway in use, injecting local anesthetics;

FIG. 4 is a distal end view from the lingual surface aspect of an exemplary intubating airway of present invention;

FIG. 5 is a proximal end view from the pharyngeal surface aspect of an exemplary intubating airway of present invention;

FIG. 6 is a bird eye view from the pharyngeal aspect of an exemplary intubating airway of present invention;

FIG. 7 is a bird eye view from the lingual aspect of an exemplary intubating airway of present invention;

FIG. 8 is a view of an exemplary intubating airway in use as partially inserted; The patient is shown in sagittal section.

FIG. 9 is a view of an exemplary intubating airway in use as fully inserted. The patient is shown in sagittal section.

FIG. 10 is a midpoint length wise cross sectional view of an exemplary intubating airway of present invention;

FIG. 11 is a prospective view of an exemplary fiber optic scope holder.

FIG. 12 is a prospective view of an exemplary intubating airway of present invention loaded with a fiber optic scope and its holder in the endotracheal tube conduit.

FIG. 13 is a midpoint length wise cross sectional view of an exemplary intubating airway of present invention showing its endotracheal tube conduit loaded with the fiber optic scope holder which is holding the fiber optic scope in its groove.

FIG. 14 is a view of an exemplary intubating airway of present invention loaded with the fiber optic scope and its holder in use as fully inserted, providing a clear unobstructed view of the larynx. At that point the operator advanced the fiber optic scope into the trachea as shown, just prior to the removal of the fiber optic scope holder and the insertion of the endotracheal tube into the trachea.

FIG. 15 is a view of an exemplary intubating airway of present invention loaded with optical stylet inside an endotracheal tube in use as fully inserted, providing the optical stylet operator with fully open airway tract and a clear view of the larynx immediately virtually converting it into a rigid C shaped optical laryngoscope.

FIG. 16 is a midpoint length wise cross sectional view of the first modified alternative embodiment of intubating airway of present invention;

FIG. 17 is a side view of the second modified alternative embodiment of intubating airway of present invention.

### DESCRIPTION OF THE PREFERRED EMBODIMENT

It is contemplated and intended that the design apply to any suitable material for making a product, the shape the body may be tubular, cylindrical or multi-edged for easy usage. For clarity reason, the examples are given in round cornered rectangular cross section shape, but an ordinary person in the art would know the variations to modify the design to make other shapes.

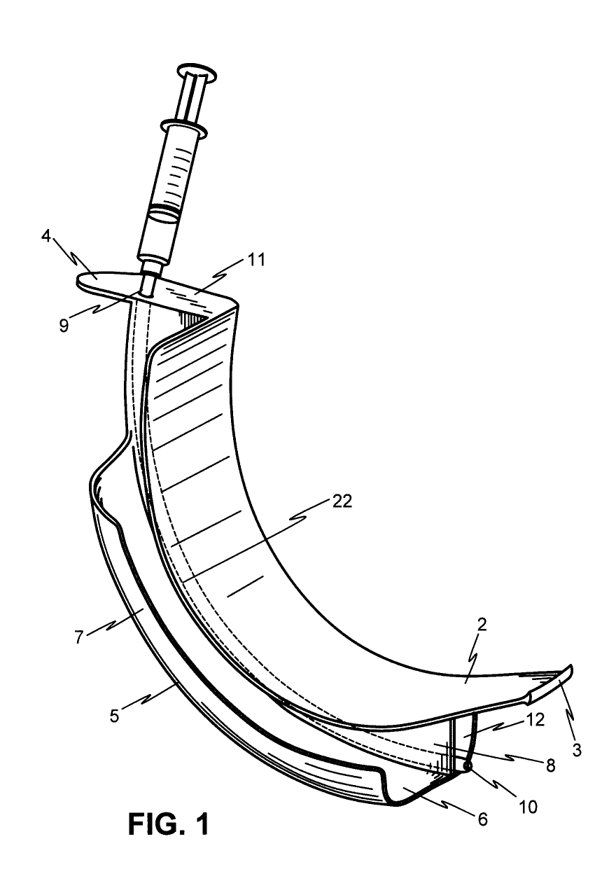

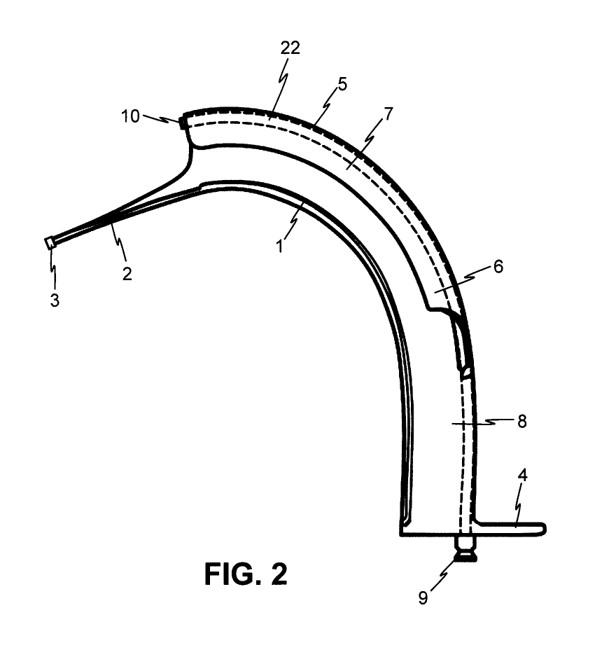

Referring to FIG. 1 and FIG. 2, there is disclosed an intubating airway 100 according to present invention. The intubating airway 100 has a curved lingual (tongue) surface 1 which is in contact with the tongue surface of a human being, and a pharyngeal surface 5 which is in contact with the posterior pharyngeal wall, back of throat. Said lingual surface 1 has a handle 4 extended from its proximal end at an angle and a tapered distal end 2 with rounded atraumatic tip 3.

Underneath said lingual surface 1, a wall 8 stands between said lingual surface 1 and a pharyngeal surface 5 perpendicularly to both lingual surface 1 and pharyngeal surface 5, whereby forming an endotracheal tube conduit 6 on one side, preferably on the right side; and a close-sided channel on the other side, preferably on the left side (left side conduit 11A).

Said left cross close-sided conduit 11A is an empty, having a closed distal end 12 and an open proximal end 11, which can accommodate a video camera.

An injection channel 22, also referred as suction channel, runs through said wall 8 along its full length, having a proximal end 9 and a distal end 10. Said handle 4 extends from said proximal end 11 towards said pharyngeal wall 5.

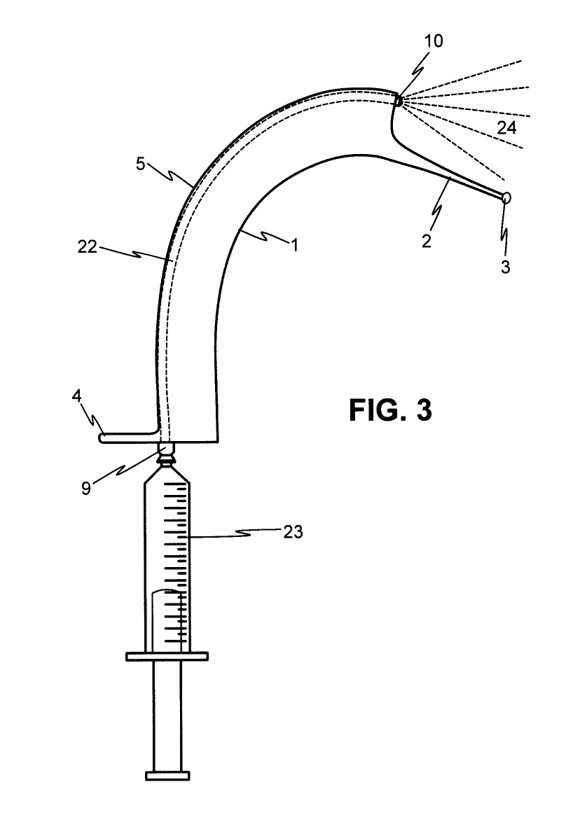

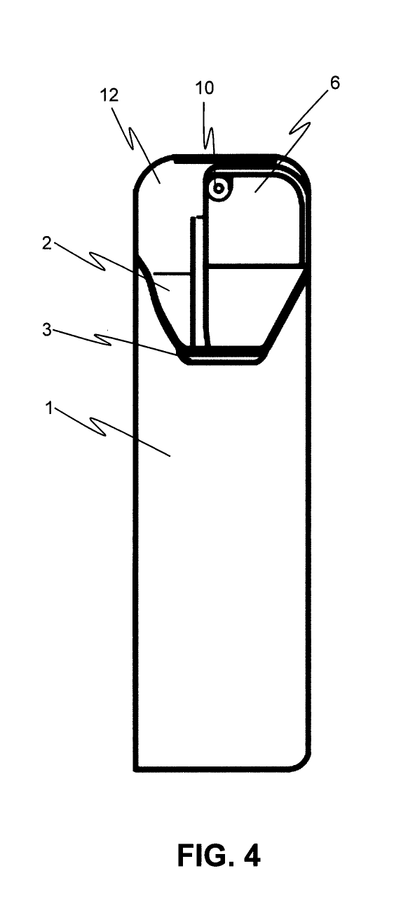

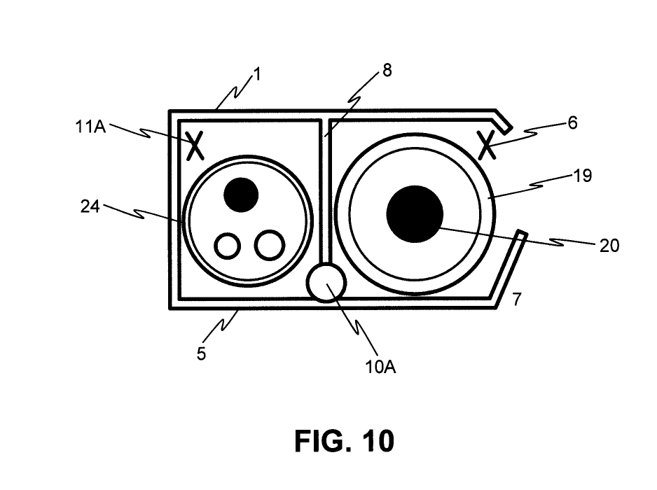

Referring to FIG. 1, FIG. 2, FIG. 3, FIG. 4 and FIG. 10 said endotracheal tube conduit 6 runs approximately proximal ⅔ along said lingual surface 1. The wall to the right of said endotracheal tube conduit 6 is partially open along its full length. This allows operator to peel away the intubating airway 100 from the endotracheal tube after tracheal intubation and remove it from the patient mouth without disturbing the endotracheal tube.

The left over exterior side wall forms a rim 7 extending upwardly from said pharyngeal surface 5. The distal ⅓ of said lingual surface 1 is free and makes the distal tapered end of the intubating airway 100 with rounded atraumatic tip 3. In fact, the intubating airway 100 has a shape of long curved spatula with two channels underneath said tongue (lingual) surface 1.

Referring to FIG. 1, FIG. 2, FIG. 3, FIG. 4 and FIG. 10 said injection channel 22 is constructed with a tube or channel embedded in said wall 8 separating the right and left conduits, close to pharyngeal surface 5, having a proximal opening 9 and a distal opening 10. It is preferably located at the bottom corner of said wall 8, which stands between the open walled right side conduit 6 and left side conduit 11A.

FIG. 3 shows injection/suction channel 22 in use for local anesthetics administration. It can be used also for suction of secretion or administration of oxygen.

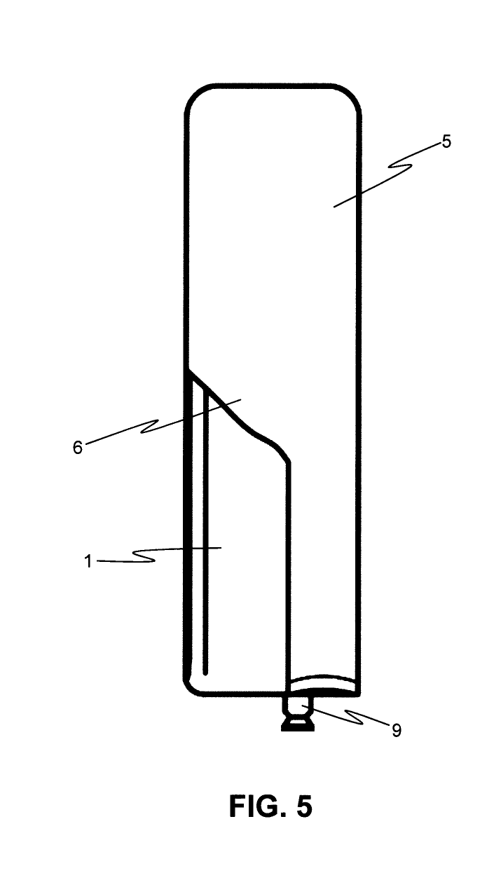

Referring to FIG. 5, the proximal end of pharyngeal surface 5 is cut to form proximal opening of endotracheal tube conduit 6. Endotracheal conduit 6 has a higher rim 7 on its pharyngeal surface 5 side than the rim on lingual surface 1 side. It is so designed that endotracheal conduit 6 may hold and guide loaded endotracheal tube, preventing the tube from sliding off track.

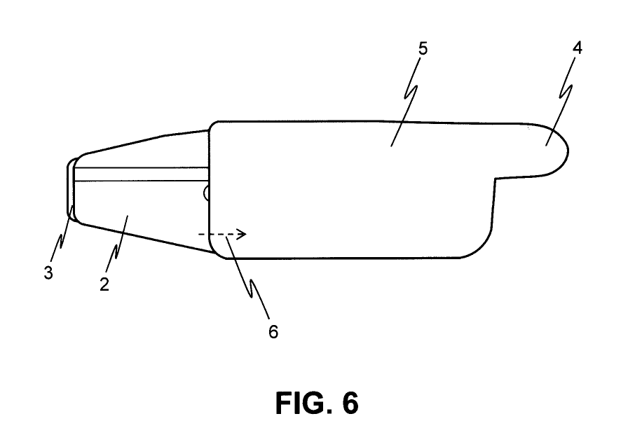

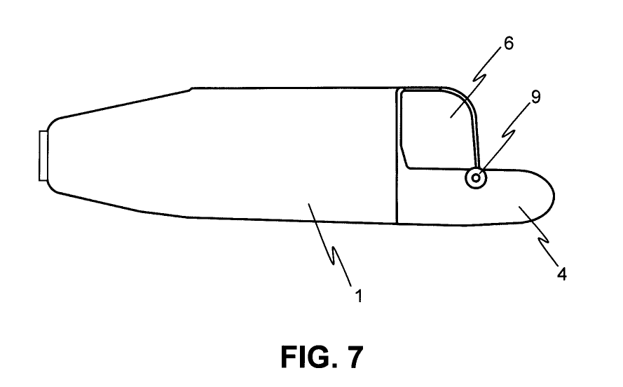

FIG. 6 and FIG. 7 show every components of intubating airway 100 in different angles, which have been described in detail above.

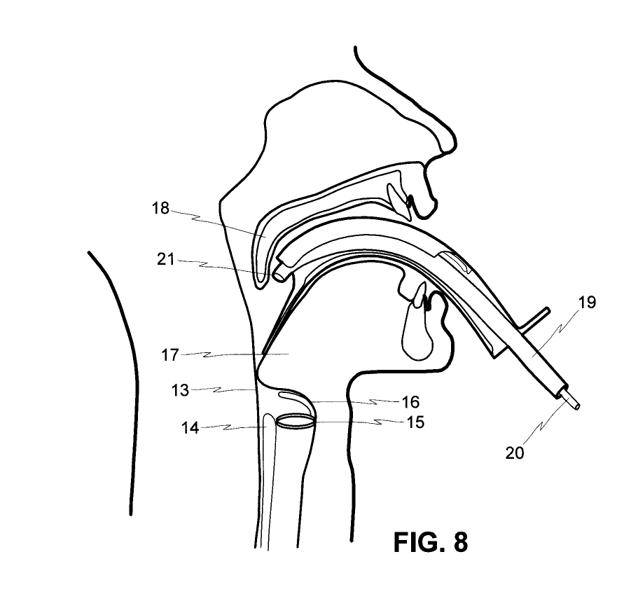

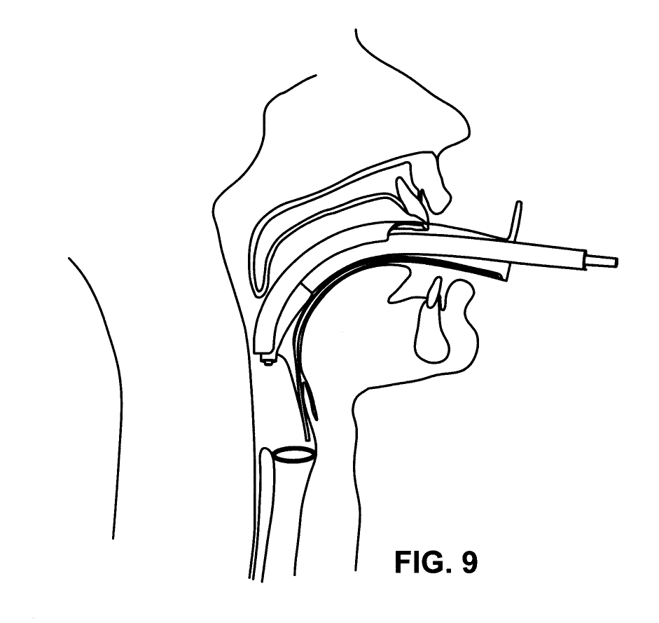

FIG. 8 and FIG. 9 show the process of inserting intubating airway 100 into patient's airway tract via the mouth. First incomplete, partial insertion as shown in FIG. 8, when performing the intubation process, a patient would lie on a steady surface facing the ceiling; intubating airway 100 is partially inserted which partially open the airway and fail to open the airway tract all the way to the larynx, leaving the distal part of the airway collapsed and obstructed. A soft thin fiber optic scope can't open the airway tract, which represents the situation with prior art devices.

FIG. 8 shows the tongue 17 relaxes and falls backward bringing it in touch with posterior pharyngeal wall 13 (back of throat), blocking the airway tract, and obstructing the view of the larynx 15 (including glottis aperture or vocal cord). This problem exists in all the commercially available and conventional brands of airways, such as Berman, Ovassapian, and Williams airways.

As shown in FIG. 9 (complete insertion) as intubating airway 100 of present invention continues to advance into patient's airway tract, it separates the tongue from the post pharyngeal wall 13 (back of throat), lifting base of the tongue 17 and epiglottis 16, therefore opening the patient's airway tract completely from mouth all the way to larynx 15, providing a full clear view of larynx 15 for a quick, and easy fiber optic or other intubation thereby.

Referring to FIG. 8 and FIG. 9, there is disclosed one process of operating an airway intubator 100 according to present invention. Endotracheal tube conduit 6 is loaded with the endotracheal tube 19, and fiber optic scope 20 is inserted inside the endotracheal tube. The distal end of endotracheal tube 19 is maneuvered to protrude just off the distal end of endotracheal tube conduit 6. Then the viewing lens with strong light of the fiber optic scope 20 is maneuvered to protrude just off the distal end of endotracheal tube 19. intubating airway 100 of present invention virtually converts the soft, thin flexible fiber optic scope into a rigid curved optical laryngoscope, which can instantly separate tissue and open the airway tract from the mouth to larynx providing a full clear view of the larynx for a quick easy intubation

Above combined unit usage makes it easy, simple and quick to use the fiber optic scope with its articulating, controllable, maneuverable tip to advance into the patient's larynx and trachea, thereby guiding the endotracheal tube along for successful intubation. Operator virtually gets a rigid C shaped laryngoscope.

More specifically, the operator will insert the intubating airway 100 with the attached endotracheal tube 19 and fiber optic scope 20 as a unit into the patient's mouth, and gradually advance it while operator looking in the eye piece of the fiber optic scope 20. Little by little, the image of soft palate 18 then the uvula then the epiglottis 16 and finally the larynx 15 come into view in turn. The airway tract will be then completely opened from the mouth to the larynx 15 for a quick easy intubation.

In the lethal situation where the patient “can't intubate and can't ventilate” due to severe airway obstruction that can't be relieved by conventional oral or nasal airways, LMA, or chin lift, jaw thrust, the specially designed airway of the present invention with its adjustable depth of insertion can easily open the airway tract from the mouth to the larynx relieving instantly the severe airway obstruction, and at the same time the operator can intubate immediately. Therefore it can be life-saving.

Again referring to FIG. 8 and FIG. 9, using the injection/suction channel and a video camera in the airway left conduit 11A, operator can install quickly and precisely local anesthetics under direct vision to anesthetize along the airway tract. First it is the soft palate 18, then the tongue 17, base of the tongue, posterior pharyngeal wall 13, piriform, fossae, and finally, epiglottis 16 and larynx 15. And as the patient takes a breath local anesthetics is then installed under vision through the open larynx 15 into the trachea, fully anesthetizing the airway tract from the mouth to the carina (the distal end of the trachea) this way the patient avoids the invasive painful transtracheal and superior laryngeal nerves injections though the neck. Awake intubation of fiber optic or other devices becomes feasible.

FIG. 10 is a cross section of airway intubator 100, showing endotracheal tube conduit 6 loaded with fiber optic scope 20 being inserted inside the endotracheal tube 19. The closed sided conduit on the left side 11A (left side conduit) can be loaded with video camera 24.

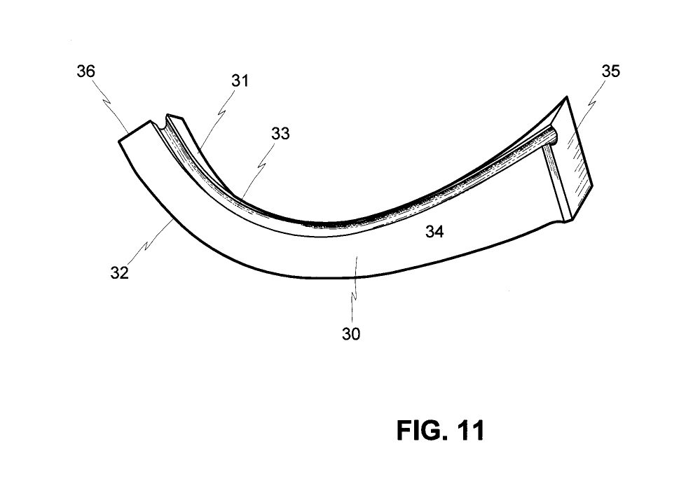

Referring to FIGS. 11, 12, 13 and 14, on the other hand, the endotracheal tube conduit 6 may accommodate a fiber optic scope 20 without inserting it into an endotracheal tube 19 in advance. To make fiber optic intubation quick and easy, the intubating airway 100 of the present invention is used to convert the thin and soft fiber optic scope into a rigid C-shaped optical laryngoscope which can separate tissue and open the airway tract completely. It is inserted and advanced into the airway tract until it provides a clear unobstructed view of the larynx. This is accomplished by having the viewing lens of the fiber optic scope temporary held stationary (in place) at the distal end of the endotracheal tube conduit 6 by a removable fiber optic scope holder 30 providing unshakeable tip and a steady viewing lens during insertion, otherwise the thin fiber optic scope will wobble inside the much larger endotracheal tube conduit 6. In the meantime the holder 30 allows easy advance of the fiber optic scope into the trachea once the larynx comes into view.

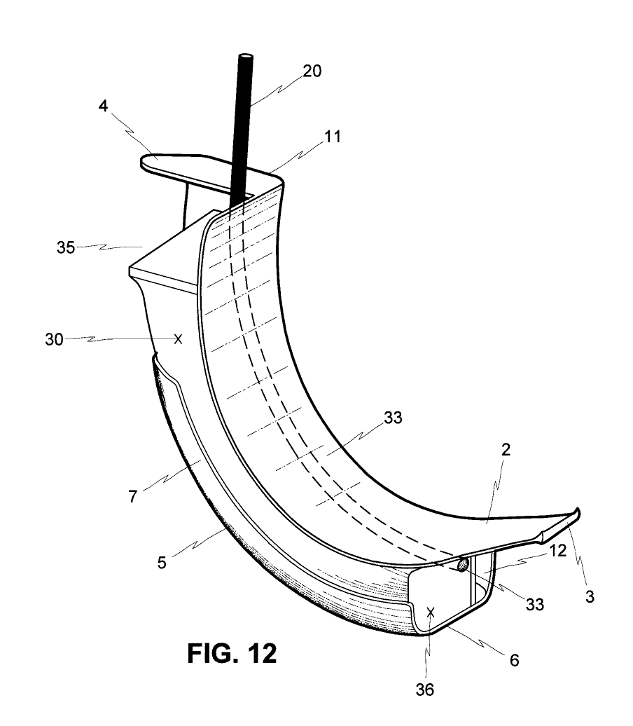

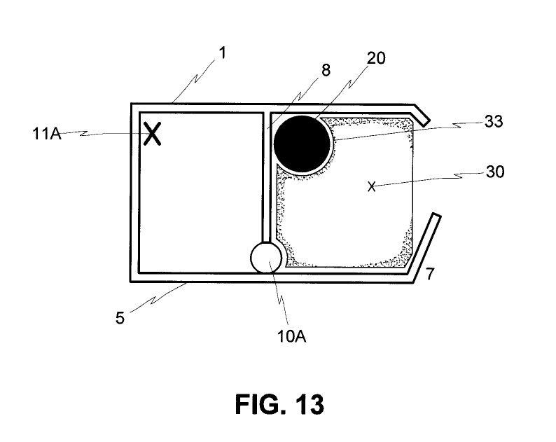

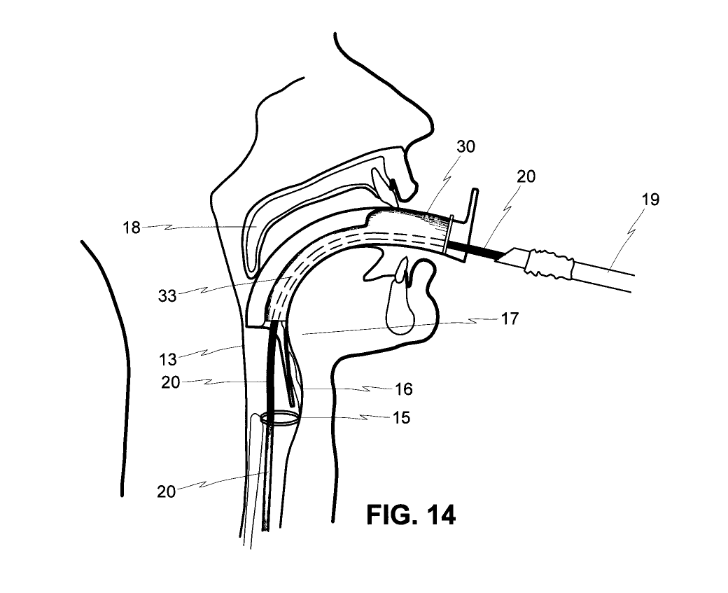

The fiber optic scope holder 30, FIG. 11 is a curved elongated rectangular flexible plastic mold of the inside of the endotracheal tube conduit 6. It has an anterior concave surface 31 in contact with the intubating airway's lingual surface 1 when it is loaded into conduit 6, a posterior convex surface 32 in contact with the pharyngeal surface 5, a left flat surface 34 in contact with said wall 8 and a right flat surface in contact with the right open sided wall. It has a proximal 35 and distal 36 ends. It has a built in groove 33 at its left anterior (lingual) corner along its full length which can be loaded with the fiber optic scope 20 and keeping its tip from wobbling as you advance the loaded intubating airway 100 into the patient airway tract. First the operator inserts the fiber optic scope holder 30 in the endotracheal tube conduit 6 until its distal end is at the distal end of the endotracheal tube conduit 6. Then the operator advances a lubricated fiber optic scope 20 into the holder's groove until its viewing lens peek off the distal end of the endotracheal conduit 6. FIG. 12 and FIG. 13. Now the operator can insert the intubating airway 100 which is loaded with the fiber optic scope 20 and its holder 30 as a unit into the patient's mouth and gradually advance it while the operator is looking into the eye piece of the fiber optic scope. The intubating airway 100 of the present invention virtually converts the soft thin fiber optic scope not only into a curved rigid optical laryngoscope but it also provides the operator with unshakeable, steady and clear view of the soft palate 18, then the uvula. then the epiglottis 16 and finally as the larynx 15 come into view, the operator can easily advance the fiber optic scope into the trachea until carina is visualized FIG. 14. Now the operator removes the fiber optic scope holder and advances the endotracheal tube 19 over the fiber optic scope 20 for a quick, easy and simple tracheal intubation. Even a first time user could intubate with high success rate. It can be used in all situations including a real emergency.

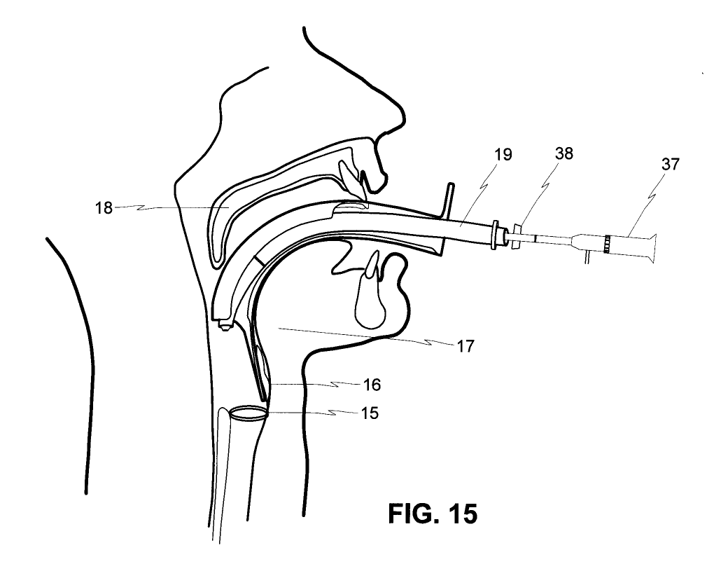

FIG. 15 illustrates another great advantage of the present invention as it shows that optical stylet intubation is quick and easy in either spontaneously breathing or anesthetized and paralyzed patients, with a very high success rate in any situation including emergency intubation even with a first time user. At the present time optical stylet intubation is very difficult, needs a lot of experience, has very low success rate especially in unpredicted difficult intubation in the anesthetized and paralyzed patients, and cannot be used reliably in a real emergency. Our present invention can facilitate optical stylet intubation in two ways:

1—Using our intubating airway 100 of the present invention to convert the optical stylet into a rigid C shaped optical or video laryngoscope which can quickly and reliably separate tissue and open the airway tract from the mouth to the larynx. The operator inserts the endotracheal tube 19 into the intubating airway's ETT tube conduit 6, aligning the distal end of the tube with the distal end of the endotracheal tube conduit 6 and inflates the cuff gently just to fix the tube inside the conduit. Then the operator inserts the optical stylet 37 and advances it until its viewing lens just peeks off the distal end of the tube and fixing it in place using the adjustable tube stop 38, virtually providing the operator with a C shaped rigid optical laryngoscope. Now the operator inserts the intubating airway 100 of the present invention loaded with the ETT tube and the optical stylet as one unit. While looking in the eye piece or the video screen the operator advances the loaded intubating airway into the patient's airway tract providing him with a clear view of the soft palate 18, then the uvula, the epiglottis 16 and finally the larynx 15. The operator deflates the ETT tube cuff and advances the styleted tube into the trachea for a successful reliable, quick and easy intubation. It can be used with great success even by first time user, in any situation including emergency intubation whether the patient breathing spontaneously or anesthetized and paralyzed.

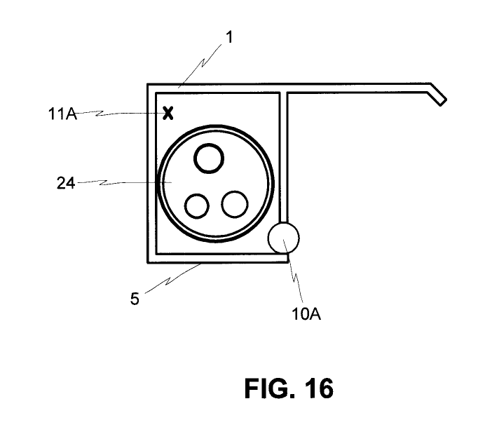

2—Using our first modified alternative of the intubating airway of the present invention FIG. 16 to quickly provide the optical stylet with an open airway from the mouth to the larynx for an easy quick intubation.

FIG. 16 shows the first modified alternative configuration of intubating airway 100 according to the present invention. Under lingual surface 1, there contains left side conduit 11A, surrounded by pharyngeal surface 5 and closed side walls on left and right side. Video camera 24 can be placed into said conduit 11A. Suction channel 10A is configured at the right corner of said conduit 11A. (There is no endotracheal conduit 6). It can be used to facilitate intubation with the optical stylets, lighted stylet, the intubation of a double lumen tube and nasal intubation when it is difficult to intubate with the conventional devices. Also it can facilitate the insertion under direct vision of a TEE probe or endoscope of the respiratory or gastrointestinal tracts. It is adapted to deliver local anesthetics under direct vision to anesthetize the airway tract quickly and precisely from the mouth to the carina prior to awake intubation.

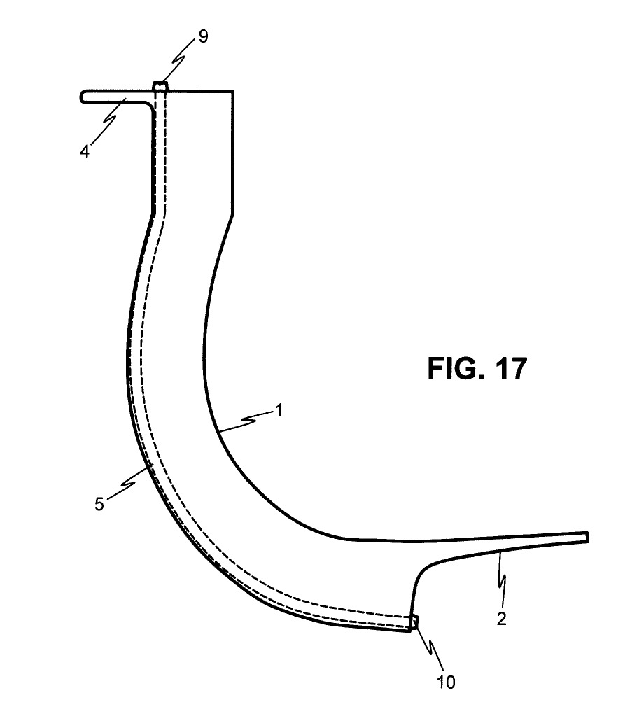

Referring to FIG. 17, there is disclosed the second modified alternative configuration of intubating airway 100 according to present invention. As seen in FIG. 12, airway intubator 100 is in the shape of an L and right angled.

The material of making the airway intubator 100 can be chosen from many conventional materials or the combination thereof, preferably from transparent plastic.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

As will be recognized by those skilled in the art, the innovative concepts described in the present application can be modified and varied over a tremendous range of applications, and accordingly the scope of patented subject matter is not limited by any of the specific exemplary teachings given. It is intended to embrace all such alternatives, modifications and variations that fall within the spirit and broad scope of the appended claims.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

The claims as filed are intended to be as comprehensive as possible, and NO subject matter is intentionally relinquished, dedicated, or abandoned.

What is claimed is:

1. An intubating airway comprising a curved lingual surface, wherein it is in contact with the tongue surface of a human being and it is 12 to 17 centimeters in length;
a pharyngeal surface, wherein it is in contact with the posterior pharyngeal wall located at the back of a human being's throat;
a handle extended from proximal end of said airway at an angle;
a tapered distal end with a traumatic tip;
a wall, which stands perpendicularly between the lingual surface and pharyngeal, whereby forming an endotracheal tube conduit on one side; and a close-sided channel on the other side.

2. An intubating airway as in claim 1, further comprising: an injection channel, also referred to as a suction channel, wherein it runs through said wall along its full length, having a proximal end and a distal end.

3. An intubating airway as in claim 2, wherein the close-sided channel is empty having a closed distal end and a open proximal end, which can accommodate a video camera and wherein a handle extends from the proximal end of the airway towards the pharyngeal wall.

4. An intubating airway as in claim 1, wherein the endotracheal tube conduit runs approximately proximal ⅔ along said lingual surface.

5. An intubating airway as in claim 2, wherein the wall to the right of the endotracheal tube conduit is partially open along its full length, which allows the airway to be peeled away from the endotracheal tube and to be removed from a patient's mouth after trachea intubation without disturbing the endotracheal tube.

6. An intubating airway as in claim 5, further comprising: a rim extending upwardly from the pharyngeal surface.

7. An intubating airway as in claim 6, wherein the distal ⅓ of the lingual surface is free and makes the distal tapered end of the intubating airway.

8. An intubating airway as in claim 2, wherein said injection channel also referred to as suction channel is preferably located at the bottom of said wall which stands between said endotracheal conduit and said close-sided conduit close to said pharyngeal surface.

9. An intubating airway as in claim 8, wherein said injection/suction channel is constructed with a tube embedded in said wall, and is configured close to said pharyngeal surface.

10. An intubating airway as in claim 9, further comprising:
an injection, also referred a suction channel, wherein it runs through said wall along its full length, having a proximal end and a distal end.

11. An intubating airway as in claim 5, wherein it has a shape of long curved spatula with two channels underneath said lingual surface.

12. An intubating airway as in claim 1, further comprising: A fiber optic scope holder with a curved elongated rectangular flexible plastic mold along the inside of the endotracheal tube conduit.

13. An intubating airway as in claim 12 with its fiber optic scope holder further comprising: an anterior concave surface that comes in contact with the lingual surface of the intubating airway of the present invention;
a posterior convex surface that comes in contact with the pharyngeal surface 5 of the intubating airway of the present invention;
a left flat surface that comes in contact with said wall 8 of the intubating airway of the present invention;
a right flat surface in contact with the right open sided wall of the intubating airway of the present invention.

14. An intubating airway as in claim 13 with its fiber optic scope holder, wherein there is a built in groove at its left anterior (lingual) corner along its full length which can accommodate a fiber optic scope

15. An intubating airway comprising a curved lingual surface, wherein it is in contact with the tongue surface of a human being:
a pharyngeal surface, wherein it is in contact with the posterior pharyngeal wall, back of a human being's throat;
a handle extended from proximal end of said lingual surface at an angle;
a tapered distal end with atraumatic tip;
right and left walls, which stands perpendicularly between said lingual surface and said pharyngeal, whereby forming a close-sided channel on the left side having a closed distal end and an open proximal end, which can accommodate a video camera and having no right side conduit

16. An intubating airway as in claim 5, wherein it has a shape of L and right angled.

17. An intubating airway as in claim 5, wherein it has an injection channel, also referred as suction channel, is constructed with a tube embedded in the wall between the right and left conduits and is configured close to said lingual surface along its full length, having a proximal and distal end

18. An intubating airway as in claim 5 wherein a handle extends forward or to the left or to the right from the proximal end.
